(19) United States
(12) Patent Application Publication
(10) Pub. No.: US 2008/0053449 A1
(43) Pub. Date: Mar. 6, 2008

(54) Respiratory Mask for Use in Endoscopy Procedures

(75) Inventors: Scott Lindblom, Charlotte, NC (US); Thomas Pope, Denver, NC (US); Thomas Pacicco, Charlotte, NC (US)

Correspondence Address: BOND, SCHOENECK & KING, PLLC, ONE LINCOLN CENTER, SYRACUSE, NY, 13202-1355, US

(73) Assignee: ENDOMASK, LLC, Charlotte, NC (US)

(21) Appl. No.: 11/848,553

(22) Filed: Aug. 31, 2007

Related U.S. Application Data
(60) Provisional application No. 60/824,163, filed on Aug. 31, 2006.

(51) Int. Cl. A62B 18/02; A61F 11/00

(52) U.S. Cl. 128/206.21; 128/868

(57) ABSTRACT

A patient respiratory mask having a shell and a patient cushion for supplying oxygen during an endoscopic or bronchoscopic procedure. The mask has a first port for accepting an endoscope, a second port for accepting oropharyngeal suction, and an optional third port for carbon dioxide monitoring. The first and second ports are covered by preferably flexible, perforated diaphragms for maintaining the seal integrity during oxygen therapy.

### CROSS-REFERENCE TO RELATED APPLICATION

The present application claims priority to U.S. Provisional Application No. 60/824,163, filed on Aug. 31, 2006.

### BACKGROUND OF THE INVENTION

1. Field of the Invention

The present invention relates to respiratory masks and, more specifically, to masks for use during endoscopy or bronchoscopic evaluation procedures.

2. Description of the Related Art

Endoscopy involves the examination of the interior of the upper alimentary canal, usually while the patient is sedated. As endoscopy may result in complications, such as apnea and hypoxia, the protocol for such procedures requires the controlled and continuous delivery of oxygen as well as the monitoring of the respiration of the patient. The devices used in combination with the controlled and continuous delivery of oxygen and monitoring of respiration usually comprise nasal cannulas and face masks. These devices are often cumbersome, ineffective for maintaining the standards of delivering the proper oxygen levels, and inadequate for monitoring the patient effectively. In addition, these devices often do not allow for the simultaneous performance of an endoscopic procedure requiring oxygen supplementation. As a result, patients are more likely to experience problems such as apnea and hypoxia that can lead to morbidity and even death.

### BRIEF SUMMARY OF THE INVENTION

It is therefore a principal object and advantage of the present invention to provide a respiratory mask that is easy to use and effective in compromised respiratory patients leading to providing oxygen supplementary at safe levels with little or no complications.

It is another object and advantage of the present invention to provide a respiratory mask that may be used during endoscopic procedures.

It is a further object and advantage of the present invention to provide a respiratory mask that is effective and safe for delivering positive pressure oxygen supplementation.

It is an additional object and advantage of the present invention to provide a respiratory mask that is adequate for monitoring the patient.

In accordance with the foregoing objects and advantages, the present invention comprises a respiratory mask that includes a port for accepting an endoscope for upper gastrointestinal (GI) tract examination and pulmonary examination. The respiratory mask includes a second port for oropharyngeal suction. The ports are positioned on the lower front portion of the mask to allow devices to enter the mouth of a patient wearing the mask. The ports include a flexible, perforated diaphragm to maintain seal integrity required for oxygen therapy purposes. The mask of the present invention may further include a third port for accepting a carbon dioxide monitoring probe. The mask of the present invention fits over the nose and mouth creating a seal and provides a ventilation port that is attached to a ventilator and a perforated diaphragm to allow passage of an endoscope. The second perforated diaphragm is used for the introduction of a suction catheter. The mask allows for oxygen therapy to be provided during endoscopy or bronchoscopy procedures without any hindrance, and provides a means for non-intubated ventilation of the patient. The mechanical ventilator allows for the simultaneous respiratory monitoring of the patient because of the seal provided by the mask.

### BRIEF DESCRIPTION OF THE DRAWINGS

The present invention will be more fully understood and appreciated by reading the following Detailed Description in conjunction with the accompanying drawings, in which:

FIG. 1 is perspective view of a respiratory mask according to the present invention.

### DETAILED DESCRIPTION OF THE INVENTION

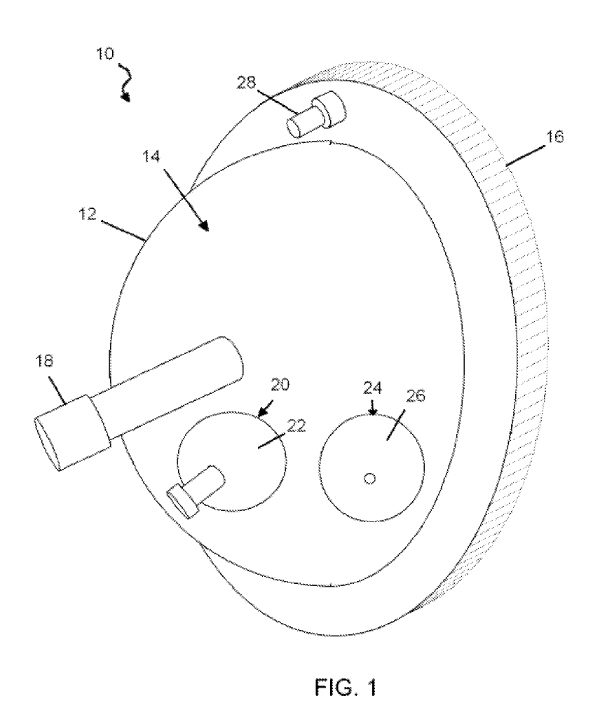

Referring now to the drawings, wherein like reference numerals refer to like parts throughout, there is seen in FIG. 1 a respiratory mask 10 according to the present invention. Mask 10 generally comprises a shell 12 defining a cavity 14 therein and an inflatable patient cushion 16 extending around the perimeter of shell 12. Mask 10 further comprises a inlet tube 18 attached to shell 12 and in fluid communication with cavity 14. It should be obvious to those of skill in the art that when placed over the face of a patient, oxygen may be delivered to mask 10 via tube 18 and inhaled by the patent.

Shell 12 includes a first port 20 formed therethrough on the lower portion of shell 12. Port 20 includes a diaphragm 22 positioned in covering relation thereto. Shell 12 further includes a second port 24 formed therethrough on the lower portion of shell 12. Second port 24 also includes second diaphragm 26 positioned in covering relation thereto. First port 20 is dimensioned to accept an endoscope for upper gastrointestinal (GI) tract examination and pulmonary examination, and second port 24 is dimensioned to accept a second port for oropharyngeal suction. Diaphragms 22 and 26 are preferably flexible, perforated diaphragms for maintaining the level of seal integrity required for oxygen therapy purposes. Shell 12 may optionally include a third port 28 for accepting a carbon dioxide monitoring probe.

What is claimed is:

1. A respiratory face mask, comprising:
a shell having a peripheral edge, an outer surface, and an inner surface defining a cavity;
an inlet tube in fluid communication with said cavity extending from the outer surface of said shell;
a first port in said shell configured to allow an endoscope to pass therethrough; and
a second port in said shell configured to allow to a suction tube to pass therethrough.

2. The mask of claim 1, further comprising a first diaphragm positioned in said first port.

3. The mask of claim 2, wherein said first diaphragm is flexible.

4. The mask of claim 3, wherein said first diaphragm is perforated.

5. The mask of claim 2, further comprising a second diaphragm positioned in said second port.

6. The mask of claim 5, wherein said second diaphragm is flexible.

7. The mask of claim 6, wherein said second diaphragm is perforated.

8. The mask of claim 5, further comprising a third port in said shell configured to allow a carbon dioxide probe to pass therethrough.

9. The mask of claim 8, further comprising a third diaphragm in said third port.

10. The mask of claim 9, wherein said third diaphragm is flexible.

11. The mask of claim 10, wherein said third diaphragm is perforated.

12. A respiratory face mask, comprising:
a shell having an upper portion, a lower portion, and peripheral edge, wherein said lower portion of said shell is proximate to the mouth of a patient when said shell is placed in covering relation to the face of said patient;
a cushion extending along said peripheral edge;
an inlet tube extending from said shell;
a first port in the lower portion of said shell; and
a second port in the lower portion of said shell.

13. The mask of claim 13, further comprising first and second diaphragms in said first and second ports, respectively.

14. The mask of claim 13, wherein said first and second diaphragms are perforated.

15. The mask of claim 14, wherein said first port is configured to accept an endoscope.

16. The mask of claim 15, wherein said second port is configured to accept a suction catheter.

17. The mask of claim 16, further comprising a third port.

18. The mask of claim 17, wherein said third port includes a diaphragm.

19. The mask of claim 18, wherein said third diaphragm is perforated.

20. The mask of claim 19, wherein said third port is configured to accept a carbon dioxide probe.
